(19) United States
(12) Patent Application Publication
(10) Pub. No.: US 2004/0227518 A1
(43) Pub. Date: Nov. 18, 2004

(54) Fuel cell voltage measuring assembly

(75) Inventors: Nathaniel Ian Joos, Toronto (CA); David Frank, Scarborough (CA)

Correspondence Address: BERESKIN AND PARR, SCOTIA PLAZA, 40 KING STREET WEST-SUITE 4000 BOX 401, TORONTO, ON, M5H 3Y2, CA

(73) Assignee: Hydrogenics Corporation, Mississauga (CA)

(21) Appl. No.: 10/778,322

(22) Filed: Feb. 17, 2004

Related U.S. Application Data
(60) Provisional application No. 60/447,289, filed on Feb. 14, 2003.

(51) Int. Cl. A61B 18/18

(52) U.S. Cl. 324/434

(57) ABSTRACT

The invention provides for measuring the voltage across an associated pair of flow field plates of each electrochemical cell in a plurality of electrochemical cells connected in series to form a stack. The invention involves (a) providing a plurality of groups of electrical contacting points for receiving an associated voltage from each of the associated pair of flow field plates for each electrochemical cell, wherein each group in the plurality of groups comprises an associated plurality of electrical contacting points; (b) electrically interconnecting the associated plurality of electrical contacting points for each group; (c) electrically insulating each group of electrical contacting points from other groups of electrical contacting points; (d) for each flow field plate, selecting an associated group from the plurality of groups of electrical contacting points, and aligning the associated group with the flow field plate, and selecting an electrical contacting point from the associated group and connecting electrically the selected electrical contacting point to the associated flow field plate, to receive the associated voltage therefrom; and (e) receiving the associated voltage from each flow field plate via the associated group.

### FIELD OF THE INVENTION

The present invention relates to a voltage measuring system for electrochemical cells.

### BACKGROUND OF THE INVENTION

A fuel cell is an electrochemical device that produces an electromotive force by bringing the fuel (typically hydrogen) and an oxidant (typically air) into contact with two suitable electrodes and an electrolyte. A fuel, such as hydrogen gas, for example, is introduced at a first electrode where it reacts electrochemically in the presence of the electrolyte to produce electrons and cations in the first electrode. The electrons are circulated from the first electrode to a second electrode through an electrical circuit connected between the electrodes. Cations pass through the electrolyte to the second electrode. Simultaneously, an oxidant, such as oxygen or air is introduced to the second electrode where the oxidant reacts electrochemically in the presence of the electrolyte and a catalyst, producing anions and consuming the electrons circulated through the electrical circuit. The cations are consumed at the second electrode. The anions formed at the second electrode or cathode react with the cations to form a reaction product. The first electrode or anode may alternatively be referred to as a fuel or oxidizing electrode, and the second electrode may alternatively be referred to as an oxidant or reducing electrode. The half-cell reactions at the first and second electrodes respectively are:

H2→2H++2e−1/2O₂+2H++2e−→H₂O

The external electrical circuit withdraws electrical current and thus receives electrical power from the fuel cell. The overall fuel cell reaction produces electrical energy as shown by the sum of the separate half-cell reactions shown in equations 1 and 2. Water and heat are typical by-products of the reaction.

In practice, fuel cells are not operated as single units. Rather, fuel cells are connected in series, either stacked one on top of the other or placed side by side. The series of fuel cells, referred to as a fuel cell stack, is normally enclosed in a housing. The fuel and oxidant are directed through manifolds in the housing to the electrodes. The fuel cell is cooled by either the reactants or a cooling medium. The fuel cell stack also comprises current collectors, cell-to-cell seals and insulation while the required piping and instrumentation are provided external to the fuel cell stack. The fuel cell stack, housing and associated hardware constitute a fuel cell module.

Various parameters have to be measured to ensure proper fuel cell stack operation and prevent damage of cell. One of these parameters is the voltage across each fuel cell in the fuel cell stack hereinafter referred to as cell voltage. Ideally, differential voltage measurement is done at the two terminals (i.e. anode and cathode) of each fuel cell in the fuel cell stack. However, since fuel cells are connected in series, and typically in large number, conventional voltage measuring systems employs a large number of contacting elements and cables to convey electronic signals representing cell voltages to a processor for analysis. Such voltage measuring systems are physically complicated and hence make cell voltage measurement often troublesome, difficult to maintain, and sometimes prohibitively expensive.

Other fuel cell voltage measuring systems employs connectors such as those commercially available from Zebra®, as disclosed in U.S. Pat. No. 6,410,176. However, such connectors have electrical contacts at fixed intervals. As can be appreciated from those skilled in the art, flow field plates of fuel cells do not have identical thickness due to manufacturing tolerance. Moreover, it is more than likely that in some fuel cell stacks, flow field plates or other fuel cell components are deliberated designed to have varied thickness. Hence such a fuel cell voltage measuring system is inadequate to work with such fuel cell stacks.

Therefore, there remains a need for a fuel cell voltage measuring system that is easy to use and maintain. Preferably, such a fuel cell voltage measuring system should have flexibility to work for fuel cell stacks having various flow field plate configurations.

### SUMMARY OF THE INVENTION

In accordance with an aspect of the invention, there is provided an assembly for measuring cell voltages for a plurality of electrochemical cells connected in series along a stack dimension to form a stack. Each electrochemical cell comprises and extends between an associated pair of flow field plates. The assembly comprises: (a) an array of electrical contacting points for receiving an associated voltage from each flow field plate for each electrochemical cell, wherein (i) the array of electrical contacting points is divided into a plurality of groups and extends along an array dimension, the array dimension being substantially alignable with the stack dimension such that the array of electrical contacting points is alignable with the plurality of electrochemical cells, (ii) each group comprises an associated plurality of electrical contacting points for aligning with the flow field plates and receiving the voltages therefrom, the associated plurality of electrical contacting points for the group being electrically interconnected, (iii) each group of electrical contacting points is electrically insulated from other groups of electrical contacting points, and (iv) the plurality of groups comprises an associated group for each flow field plate for receiving the associated voltage of only that flow field plate; and, (v) for each group, the associated plurality of electrical contacting points are spaced from one another to accommodate variation in positioning of the flow field plates; and (b) an electrical connection means for receiving the associated voltage from each flow field plate via the associated group, the electrical connection means comprising an associated connector for each group for separately receiving voltage signals from the group.

In accordance with a second aspect of the invention, there is provided a multi-cell electrochemical device assembly comprising (a) a plurality of electrochemical cells connected in series along a stack dimension to form a stack, wherein each electrochemical cell comprises and extends between an associated pair of flow field plates; (b) an array of electrical contacting points for receiving an associated voltage from each of the associated pair of flow field plates for each electrochemical cell, wherein (i) the array of electrical contacting points is divided into a plurality of groups and extends along an array dimension, the array dimension being substantially alignable with the stack dimension such that the array of electrical contacting points is alignable with the plurality of electrochemical cells, (ii) each group comprises an associated plurality of electrical contacting points for aligning with the flow field plates and receiving the voltages therefrom, the associated plurality of electrical contacting points for the group being electrically interconnected, (iii) each group of electrical contacting points is electrically insulated from other groups of electrical contacting points, (iv) the plurality of groups comprises an associated group for each flow field plate for receiving the associated voltage of only that flow field plate; and, (v) for each group, the associated plurality of electrical contacting points are spaced from one another to accommodate variation in positioning of the flow field plates; and (c) an electrical connection means for receiving the associated voltage from each flow field plate via the associated group, the electrical connection means comprising an associated connector for each group for separately receiving voltage signals from the group.

In accordance with a third aspect of the invention, there is provided a method of measuring the voltage across an associated pair of flow field plates of each electrochemical cell in a plurality of electrochemical cells connected in series to form a stack. The method comprises: (a) providing a plurality of groups of electrical contacting points for receiving an associated voltage from each of the associated pair of flow field plates for each electrochemical cell, wherein each group in the plurality of groups comprises an associated plurality of electrical contacting points; (b) electrically interconnecting the associated plurality of electrical contacting points for each group; (c) electrically insulating each group of electrical contacting points from other groups of electrical contacting points; (d) for each flow field plate, selecting an associated group from the plurality of groups of electrical contacting points, and aligning the associated group with the flow field plate, and selecting an electrical contacting point from the associated group and connecting electrically the selected electrical contacting point to the associated flow field plate, to receive the associated voltage therefrom; and (e) receiving the associated voltage from each flow field plate via the associated group.

### BRIEF DESCRIPTION OF THE DRAWINGS

For a better understanding of the present invention and to show more clearly how it may be carried into effect, reference will now be made, by way of example, to the accompanying drawings which show a preferred embodiment of the present invention and in which:

FIG. 1 is a schematic view of a fuel cell voltage measuring assembly in accordance with a first embodiment of the present invention, mounted on a fuel cell stack;

FIG. 2 is a schematic perspective view of the fuel cell voltage measuring assembly in accordance with the first embodiment of the present invention;

FIG. 3 is an enlarged view of portion A in FIG. 1;

FIG. 4 is a sectional view along B-B line in FIG. 3;

FIG. 5 is a first perspective view of a mounting frame in accordance with a second embodiment of the present invention;

FIG. 6 is another perspective view of the mounting frame in accordance with a second embodiment of the present invention; and

FIG. 7 is a schematic view of a fuel cell voltage measuring assembly in accordance with the second embodiment of the present invention, mounted on a fuel cell stack.

### DETAILED DESCRIPTION OF THE PREFERRED EMBODIMENT OF THE INVENTION

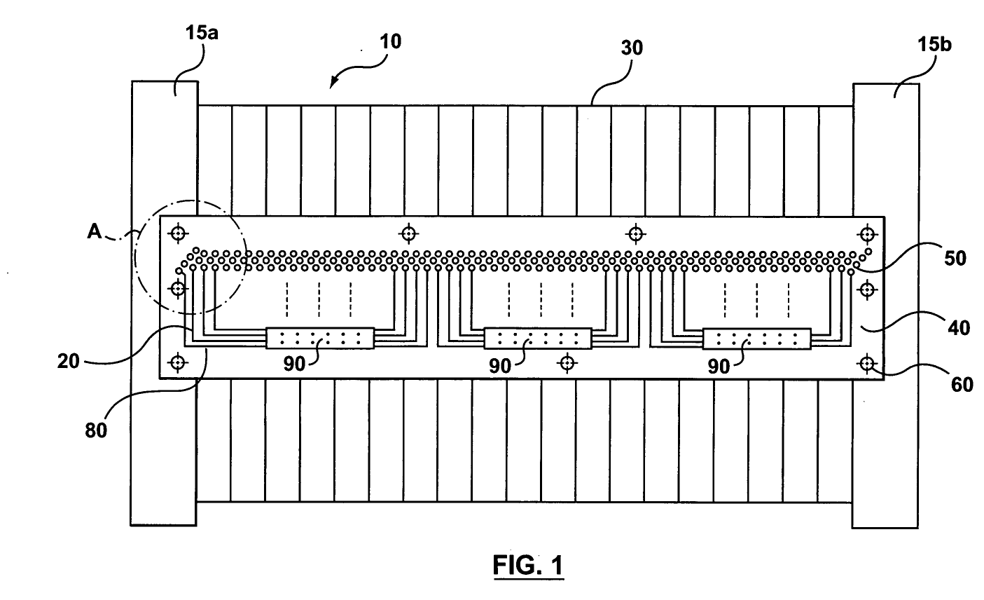

Referring first to FIG. 1, this shows a fuel cell stack 10 comprising a plurality of fuel cells 30 stacked in series. Taking Proton Exchange Membrane (PEM) fuel cells as an example, each fuel cell typically consists of two flow field plates for supplying reactants, namely fuel and oxidant to a proton exchange membrane disposed therebetween. Each fuel cell typically generates a voltage of about 0.6 to 1.0 volts. Cell voltages are usually measured at two flow field plates of each fuel cell 30.

A fuel cell voltage measuring assembly 20 extends parallel to the longitudinal direction of the fuel cell stack 10, and is mounted, at two ends thereof, on the side faces of two end plates 15a and 15b of the fuel cell stack 10. The fuel cell voltage measuring assembly 20 generally comprises a Printed Circuit Board (PCB) 40 and a plurality of probes 70 (FIG. 4) detachably soldered on the PCB 40 in a plurality of pin holes 50. For clarity, probes 70 are not shown in FIG. 1 but can be clearly seen from FIG. 4, which will be explained in detail below. Conventional techniques can be utilized for soldering probes to the PCB 40.

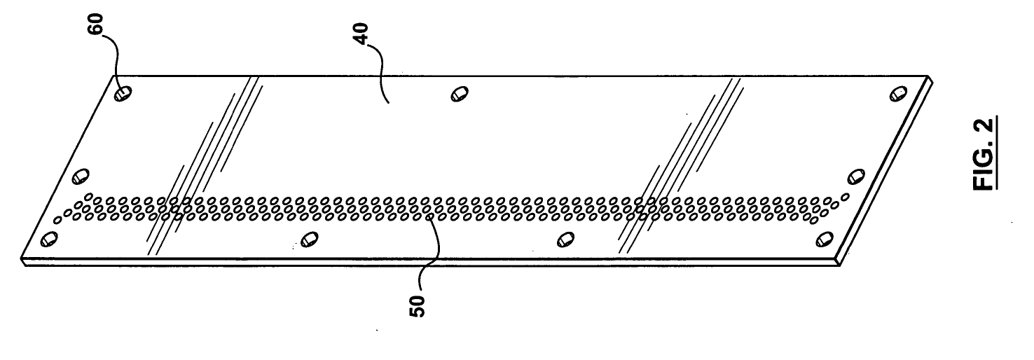

The pin holes 50 are formed in a plurality of groups. In FIGS. 1 and 2, each pin hole group consists of three pin holes 50. Pin holes 50 in each group are electrically connected with one another. Each group of pin holes 50 is not in electrical connection with any other group of pin holes 50. Each group of pin holes 50 is electrically connected to a multi-pin connector 90 soldered on the PCB 40 via printed circuits 80. For illustration only, FIG. 1 shows three such connectors 90. As can be understood to those skilled in the art, the multi-pin connectors 90 are used to provide electrical connection with external circuits and/or processor for analysis of fuel cell voltages measured by the present assembly.

Groups of pin holes are provided on the PCB 40 along the longitudinal direction of the fuel cell stack 10. Probes 70 are disposed in the pin holes 50 to measure cell voltages. During cell voltage measurement, probes 70 are in contact with flow field plates of the fuel cells 30. Probes 70 can be spring loaded to press against flow field plates. Various types of probes can be used in the present invention. An example of such spring loaded probes is commercially available from Interconnect Devices, Inc., Kansas City, U.S. As shown in FIG. 4, a probe 70 generally consists of a contacting portion 72 for contacting fuel cell flow field plates and a housing portion 74. A spring 76 is disposed within the housing portion 74 to bias the contacting portion 72 against flow field plates. Both the contacting portion 72 and the housing portion 74 are electrically conductive and the housing portion 74 is soldered on the PCB 40. In this way, electrical signals representing fuel cell voltages are conveyed to the multi-pin connectors 90 which in turn convey the signals to external processor for analysis.

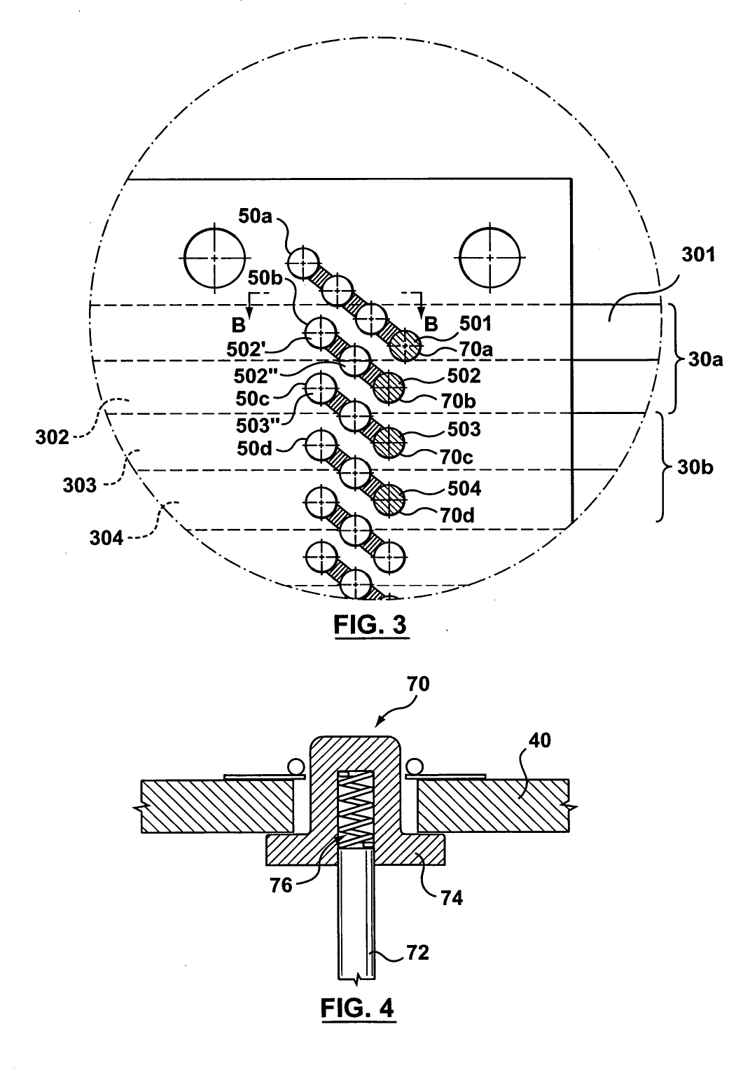

As can be appreciated from those skilled in the art, flow field plates of fuel cells do not have identical thickness due to manufacturing tolerances. Moreover, in some fuel cell stacks, flow field plates or other fuel cell components may be deliberated designed to have varied thickness. A fuel cell voltage measuring system should have the adaptability to take voltage measurements from such fuel cell plates or components. In the present invention, a probe 70 can be disposed in any one pin hole of each group of pin holes 50. The relative position of pin holes 50 within each group can be arbitrary; however, it is preferred that they be offset along the longitudinal direction of the fuel cell stack 10, as can best be seen in FIG. 3. In this way, the probes 70 can be selectively disposed in pin holes to match the longitudinal positions of fuel cell flow field plates. In an example shown in FIG. 3, probes 70a, 70b, 70c and 70d are respectively disposed in pin holes 501, 502, 503 and 504 of pin hole groups 50a, 50b, 50c and 50d, to measure the voltage of flow field plates 301, 302, 303 and 304 of fuel cell 30a and 30b. If the thickness of any of the flow field plates 301-304 is different, for example, if flow field plate 301 is thinner than shown, then probe 70b may have to be disposed in pin hole 502′ in order to measure voltage of flow field plate 302. If flow field plate 302 is also thinner than shown, then probe 70c may have to be disposed in pin hole 503′.

Optionally, probes 70 can be disposed in more than one pin hole 50 within each group—even in all of the pin holes 50—provided that some means is provided for making sure that different probes 70 in the same group of pin holes 50 can be disabled in cases where they abut a flow field plate whose voltage is not to be measured at that group of pin holes. For example, referring to FIG. 3, probes 70b could be mounted in pinholes 502, 502′ and 502′. However, in such embodiments, it would be necessary to disable the probes 70b in pinholes 502′ and 502′, if pin hole group 50b is intended to measure the voltage of flow field plate 302 and not that of flow field plate 301. Alternatively, it would be necessary to disable the probes 70b in pinholes 502 and 502′, if pin hole group 50b is intended to measure the voltage of flow field plate 301 and not that of flow field plate 302. A probe 70 could be disabled, for example, by disabling its spring such that it no longer extends to press against the surface of the abutting flow field plate.

One group of pin holes is provided for each and every fuel cell flow field plate. Of course, there may be some flexibility in how the groups of pin holes are used to ensure that there is a group of pin holes allocated for each flow field plate as pin holes from more than one group may be positioned next to the same flow field plate. For example, referring to FIG. 3, the voltage of flow field plate 302, could be measured by probe 70b in pin hole 502 of pin hole group 50b, or by probe 70c in pin hole 503′ of pin hole group 50c. On the other hand, one group of pin holes may be used to measure voltages of different flow field plates. For example, a probe can be disposed in 502′ to measure voltage of flow field plate 301, or in 502 to measure voltage of flow field plate 302. However, each group of pin holes should be used to measure the voltage of only one flow field plate at a time as otherwise the voltage signals from two different flow field plates may be confused.

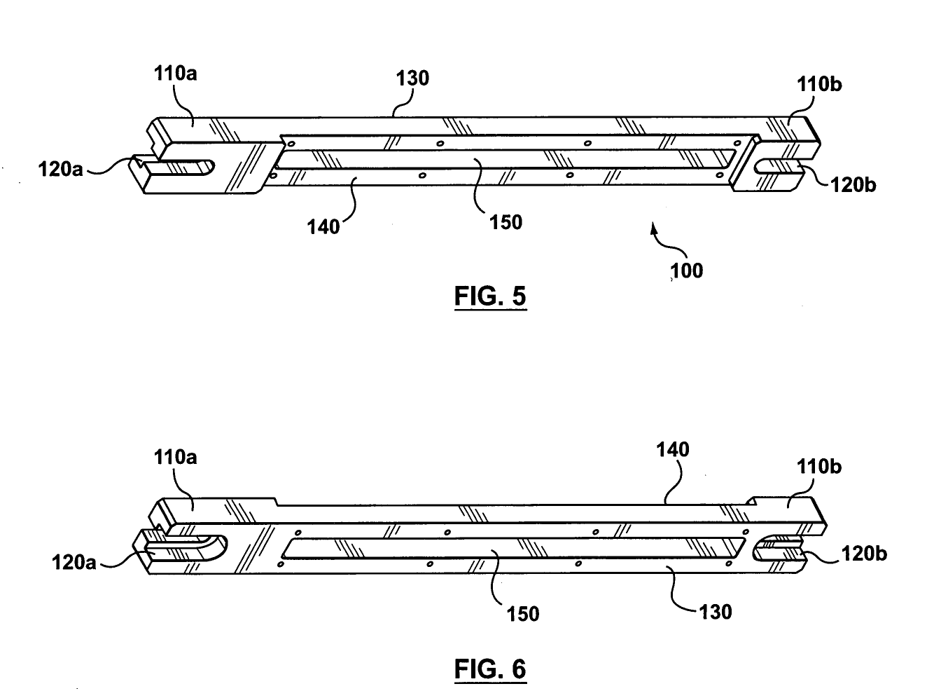

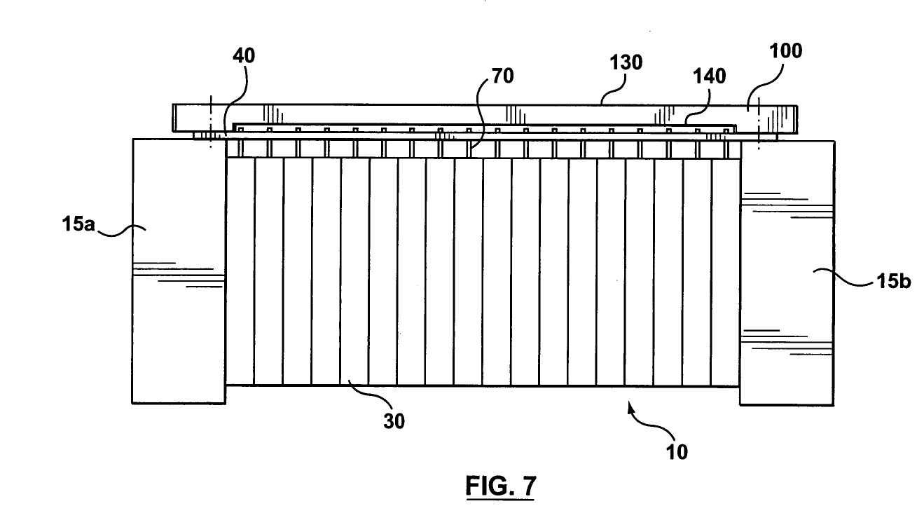

Now referring to FIGS. 5-7, these show a second embodiment of the present invention. In this embodiment, a mounting frame 100 is used to secure the PCB 40 on to the end plates 15a and 15b of the fuel cell stack 10 and protect the PCB 40. The mounting frame 100 has two open ends 110a and 110b, each having a slot 120a and 120b. Screws (not shown) can pass through these slots 120a, 120b and screw holes 60 of the PCB 40 (FIG. 1) to mounting the PCB 40 onto the end plates 15a and 15b. The mounting frame co-extends with the PCB 40 along the longitudinal direction of the fuel cell stack 10, and when mounted on the fuel cell stack 10, has an upper face 130 and a lower face 140. The two end portions 110a and 110b protrude from the lower face 140 and press against the PCB 40. A long slot 150 is provided in the mounting frame 100 to accommodate protrusion of probes 70 above the PCB 40.

The present invention enables easy fuel cell voltage measurement and allows for such measurement to be done for various designs of fuel cell stacks. It should be appreciated that the present invention is intended not only for measuring the voltages of individual fuel cells, in fuel cell stacks, but also for measuring the voltages in any kind of electrochemical cell or multi-cell battery formed by connecting individual cells in series. The present invention can also be used to measure the voltage of a single cell, a battery, a battery bank or an electrolyser.

It should be further understood that various modifications can be made, by those skilled in the art, to the preferred embodiments described and illustrated herein, without departing from the present invention, the scope of which is defined in the appended claims.

1. An assembly for measuring cell voltages for a plurality of electrochemical cells connected in series along a stack dimension to form a stack, wherein each electrochemical cell comprises and extends between an associated pair of flow field plates, the assembly comprising:
(a) an array of electrical contacting points for receiving an associated voltage from each flow field plate for each electrochemical cell, wherein
(i) the array of electrical contacting points is divided into a plurality of groups and extends along an array dimension, the array dimension being substantially alignable with the stack dimension such that the array of electrical contacting points is alignable with the plurality of electrochemical cells,
(ii) each group comprises an associated plurality of electrical contacting points for aligning with the flow field plates and receiving the voltages therefrom, the associated plurality of electrical contacting points for the group being electrically interconnected,
(iii) each group of electrical contacting points is electrically insulated from other groups of electrical contacting points, and
(iv) the plurality of groups comprises an associated group for each flow field plate for receiving the associated voltage of only that flow field plate; and,
(v) for each group, the associated plurality of electrical contacting points are spaced from one another to accommodate variation in positioning of the flow field plates; and
(b) an electrical connection means for receiving the associated voltage from each flow field plate via the associated group, the electrical connection means comprising an associated connector for each group for separately receiving voltage signals from the group.

2. The assembly as defined in claim 1 further comprising a plurality of electrical contacting members for contacting the plurality of flow field plates, wherein for each flow field plate, the associated group is positionable such that an associated active electrical contacting point in the associated group is adjacent to the flow field plate; and the associated active electrical contacting point in the associated group is conductively linked to an associated electrical contacting member in the plurality of electrical contacting members, the associated electrical contacting member being operable to contact the flow field plate.

3. The assembly as defined in claim 2 wherein the associated plurality of electrical contacting points in each group are offset with respect to one another along the array dimension.

4. The assembly as defined in claim 2 further comprising a printed circuit board, wherein
the array of electrical contacting points is an array of holes in the printed circuit board, and each electrical contacting point in the array of electrical contacting points is a hole in the array of holes;
for each group of holes in the array of holes, the associated connector comprises an associated printed circuit on the printed circuit board; and
the plurality of electrical contacting members is a plurality of extendable probes, each extendable probe being mountable in a hole.

5. The assembly as defined in claim 4 wherein the printed circuit board is detachably mountable on the stack to hold the plurality of extendable probes against the stack.

6. The assembly as defined in claim 4 wherein the printed circuit board is mountable on the stack and each extendable probe in the plurality of extendable probes is biased to extend to hold the extendable probe against the stack.

7. An assembly as claimed in claim 4 further comprising a mounting frame attached to the printed circuit board, the mounting frame being detachably mountable on the two ends of the stack to secure the printed circuit board on the stack.

8. An assembly as claimed in claim 4, wherein the mounting frame comprises a slot extending along the array dimension to accommodate the plurality of extendable probes protruding from the printed circuit board.

9. A multi-cell electrochemical device assembly comprising
(a) a plurality of electrochemical cells connected in series along a stack dimension to form a stack, wherein each electrochemical cell comprises and extends between an associated pair of flow field plates;
(b) an array of electrical contacting points for receiving an associated voltage from each of the associated pair of flow field plates for each electrochemical cell, wherein
(i) the array of electrical contacting points is divided into a plurality of groups and extends along an array dimension, the array dimension being substantially alignable with the stack dimension such that the array of electrical contacting points is alignable with the plurality of electrochemical cells,
(ii) each group comprises an associated plurality of electrical contacting points for aligning with the flow field plates and receiving the voltages therefrom, the associated plurality of electrical contacting points for the group being electrically interconnected,
(iii) each group of electrical contacting points is electrically insulated from other groups of electrical contacting points,
(iv) the plurality of groups comprises an associated group for each flow field plate for receiving the associated voltage of only that flow field plate; and,
(v) for each group, the associated plurality of electrical contacting points are spaced from one another to accommodate variation in positioning of the flow field plates; and
(c) an electrical connection means for receiving the associated voltage from each flow field plate via the associated group, the electrical connection means comprising an associated connector for each group for separately receiving voltage signals from the group.

10. The assembly as defined in claim 9 further comprising a plurality of electrical contacting members for contacting the plurality of flow field plates, wherein for each flow field plate,
the associated group is positioned such that an associated active electrical contacting point in the associated group abuts the flow field plate; and,
the associated active electrical contacting point is conductively linked to an associated electrical contacting member in the plurality of electrical contacting members for contacting the flow field plate.

11. The assembly as defined in claim 10 wherein the associated plurality of electrical contacting points in each group are offset with respect to one another along the array dimension.

12. The assembly as defined in claim 10 further comprising a printed circuit board, wherein
the array of electrical contacting points is an array of holes in the printed circuit board, and each electrical contacting point in the array of electrical contacting points is a hole in the array of holes;
for each group of holes in the array of holes, the associated connector comprises an associated printed circuit on the printed circuit board; and
the plurality of electrical contacting members is a plurality of extendable probes, each extendable probe being mountable in a hole.

13. The assembly as defined in claim 12 wherein the printed circuit board is detachably mounted on the stack to hold the plurality of extendable probes against the stack.

14. The assembly as defined in claim 12 wherein the printed circuit board is mounted on the stack and each extendable probe in the plurality of extendable probes is biased to extend toward the stack to hold the plurality of extendable probes against the stack.

15. An assembly as claimed in claim 12 further comprising a mounting frame attached to the printed circuit board, the mounting frame being detachably mountable on the two ends of the stack to secure the printed circuit board on the stack.

16. An assembly as claimed in claim 15, wherein the mounting frame comprises a slot extending along the array dimension to accommodate the plurality of extendable probes protruding from the printed circuit.

17. A method of measuring the voltage across an associated pair of flow field plates of each electrochemical cell in a plurality of electrochemical cells connected in series to form a stack, the method comprising:
(a) providing a plurality of groups of electrical contacting points for receiving an associated voltage from each of the associated pair of flow field plates for each electrochemical cell, wherein each group in the plurality of groups comprises an associated plurality of electrical contacting points;
(b) electrically interconnecting the associated plurality of electrical contacting points for each group;
(c) electrically insulating each group of electrical contacting points from other groups of electrical contacting points;
(d) for each flow field plate, selecting an associated group from the plurality of groups of electrical contacting points, and aligning the associated group with the flow field plate, and selecting an electrical contacting point from the associated group and connecting electrically the selected electrical contacting point to the associated flow field plate, to receive the associated voltage therefrom; and
(e) receiving the associated voltage from each flow field plate via the associated group.

18. The method as defined in claim 17 further comprising, for each group, spacing the associated plurality of electrical contacting points from one another to accommodate variation in positioning of the flow field plates.

19. The method as defined in claim 18 wherein, for each group, the step of spacing the associated plurality of electrical contacting points from one another comprises offsetting the associated plurality of electrical contacting points with respect to one another along a longitudinal dimension of the stack.
